(19) United States
(12) Patent Application Publication
(10) Pub. No.: US 2023/0002314 A1
(43) Pub. Date: Jan. 5, 2023

(54) IMPROVED PROCESS FOR PREPARING UNSYMMETRICAL DIALKYL SULFIDES

(71) Applicant: SALTIGO GmbH, Leverkusen (DE)

(72) Inventor: Andre Grossmann, Cologne (DE)

(73) Assignee: SALTIGO GmbH, Leverkusen (DE)

(21) Appl. No.: 17/780,593

(22) PCT Filed: Nov. 27, 2020
(86) PCT No.: PCT/EP2020/083655

§ 371 Date: May 27, 2022

(30) Foreign Application Priority Data
Nov. 28, 2019 (EP) 19212191.1

(51) Int. Cl. C07C 319/14

(52) U.S. Cl. CPC C07C 319/14

(57) ABSTRACT

The invention relates to an improved process for preparing unsymmetrical dialkyl sulfides by reacting a monoalkyl sulfide with at least one alkyl halide in the presence of base.

The invention relates to an improved process for preparing unsymmetrical dialkyl sulfides by reacting a monoalkyl sulfide with at least one alkyl halide in the presence of base.

Such unsymmetrical dialkyl sulfides are reagents from which sulfonium salts can be generated, which in turn may be used to advantage in epoxidation reactions, such as the Corey-Chaykovsky reaction. Intermediates can be obtained via these epoxidation reactions which may be used in the synthesis of pharmaceutical active ingredients, for example fluconazole, or active ingredients in plant protection, for example cyproconazole.

Chemical methods are already known for the production of unsymmetrical dialkyl sulfides. For example, C. Kimura et al., Yuki Gosei Kagaku Kyokaishi (1977), 35(8), 669-71, discloses the preparation of ethyl octyl sulfide by reacting octanethiol with ethyl bromide in the presence of a phase transfer catalyst, for example tridodecylethylammonium bromide, and in the presence of sodium hydroxide. Here, the two reactants, aqueous sodium hydroxide solution and phase transfer catalyst, are combined and reacted at 0 to 30° C. with vigorous mixing.

JP2010285408A discloses a process for preparing dodecyl methyl sulfide from dodecylthiol and sodium methanethiolate in the presence of the phase transfer catalyst tetrabutylammonium bromide at temperatures of 80° C. in yields of at least 95%.

JP2010285378A and EP2441751 A1 disclose a process for preparing dialkyl sulfides, for example ethyl isopropyl sulfide, from alkyl halides, for example 2-bromopropane, and alkyl mercaptan salts, for example sodium ethanethiolate, obtained from ethanethiol and sodium hydroxide solution, in the presence of base, for example sodium hydroxide, a reducing agent, for example sodium borohydride, and phase transfer catalyst, for example tetrabutylammonium bromide, at temperatures from 0 to 50° C. in yields of at least 95%. Here, the reducing agent serves to avoid the formation of alkyl disulfides.

EP2351735 A1 discloses a process for preparing dialkyl sulfides, for example butyl isobutyl sulfide, from alkyl halides, for example 1-bromo-2-methylpropane, and alkyl mercaptan salts, for example sodium butanethiolate, obtained from butanethiol and sodium hydroxide solution, in the presence of base, for example sodium hydroxide, a reducing agent, for example sodium borohydride, and phase transfer catalyst, for example tetrabutylammonium bromide, at temperatures from 0 to 50° C. These are not isolated, but rather directly oxidized to the corresponding sulfones.

Although these processes afford the desired products in very good yields, they have decisive disadvantages when used on a larger scale, for example a 20 to 100 liter scale. If, for example, as in C. Kimura et al., all the reactants of the reaction, i.e. alkyl sulfide, alkyl halide, base and phase transfer catalyst, are combined all at once at room temperature or at elevated temperature, for example 50° C., the resulting heat of reaction may easily bring the temperature of the reaction mixture to temperatures above 100° C. In addition, with this procedure, the salt of the base and of the alkyl sulfide may form and crystallize out to a greater extent due to a high excess of base. Both can possibly be uncontrollable on an industrial scale. On the other hand, a reaction of an alkyl halide with a sodium alkyl thiolate, which is commercially available or prepared before the reaction, and/or the addition of further reactants such as reducing agents, leads to increased production costs and/or to increased process expenditure. The increase in the production costs and/or the complexity of the process also results from the use of solvents other than water, which may have to be disposed of as waste after the reaction.

Therefore, there was still the need for and thus the object of an inexpensive, low-waste and simple process for preparing unsymmetrical dialkyl sulfides, which on the one hand affords the high yields of the processes of the prior art even on an industrial scale and on the other hand does not have their disadvantages.

An improved process for preparing unsymmetrical dialkyl sulfides of the formula (I) has now been found,

R¹—S—R² (I),

comprising the reaction of an alkyl sulfide of the formula (II),

H—S—R² (II),

with an alkyl halide of the formula (III),

R¹—X (III),

wherein in the formulae (I), (II) and (III)
R¹ is a C₁—C₃-alkyl radical, preferably methyl,
R² is a C₄—C₁₂ alkyl radical,
X is halogen, preferably chloride, bromide, iodide, particularly preferably chloride, at least in the presence of a base, preferably in the presence of an alkali metal hydroxide, wherein a reaction mixture is formed, and the temperature of the reaction mixture during the reaction is in a range from 15 to 100° C., preferably from 15 to 50° C., particularly preferably from 25 to 40° C.

In the process according to the invention, the reaction is preferably carried out in the presence of water and a phase transfer catalyst, preferably a quaternary ammonium salt of the formula (IV),

R³R⁴₃N⁺Y⁻ (IV),

in which
R³ is hydrogen or methyl, preferably methyl,
R⁴ is a C₄—C₁₂ alkyl radical, preferably octyl and/or decyl,
X is halogen, preferably chloride, bromide or iodide.
R² in formula (I) and formula (II) is preferably n-butyl, n-pentyl, n-hexyl, n-heptyl, n-octyl, n-nonyl, n-decyl, n-undecyl or n-dodecyl. R² in formula (I) and formula (II) is particularly preferably n-butyl.

In the process according to the invention, preferably to a mixture of the alkyl sulfide of the formula (II) and the phase transfer catalyst, preferably the quaternary ammonium salt of the formula (IV), and optionally water, is simultaneously added base, preferably the alkali metal hydroxide, and the alkyl halide of the formula (III).

The temperature of the reaction mixture is preferably not regulated by external heat supply, but rather by the rate of addition of the base, preferably the alkali metal hydroxide. Since the reaction of the base, preferably the alkali metal hydroxide, with the alkyl sulfide of the formula (II) to form the corresponding salt of the alkyl sulfide of the formula (II) is exothermic, the temperature of the reaction mixture can be easily and securely adjusted and maintained at the required temperature range of 15 to 100° C., preferably of 15 to 50° C., particularly preferably of 25 to 40° C., by the rate at which the base, preferably the alkali metal hydroxide, is added.

It is advantageous for the process according to the invention if, at the beginning of the reaction, from 0.01 to 0.05 mol, preferably 0.02 to 0.04 mol, of base, preferably alkali metal hydroxide, based on 1.0 mol of alkyl sulfide of the formula (II), are added to the mixture of alkyl sulfide of the formula (II), optionally phase transfer catalyst, preferably the quaternary ammonium salt of the formula (IV), and optionally water, before alkyl halide of the formula (III) is added to the reaction mixture. As a result, right from the start of the reaction, a proportion of the alkyl sulfide of the formula (II) is present as salt of the base, preferably of the alkali metal hydroxide, which can react directly with added alkyl halide of the formula (III).

In the further course of the reaction, base, preferably alkali metal hydroxide, is added to the reaction mixture. In this case, it is advantageous for the process according to the invention if the alkyl halide of the formula (III), particularly preferably in the gaseous state, is added to the reaction mixture together with the base, preferably the alkali metal hydroxide. The alkyl halide of the formula (III) is particularly preferably added to the reaction mixture together with the base, preferably with the alkali metal hydroxide, in such a way that sufficient alkyl halide of the formula (III) is present in the reaction system at all times during the reaction, so that conversion to the dialkyl sulfide of the formula (I) takes place as rapidly as possible. In a preferred embodiment, this is achieved in that the reaction takes place in a closed reactor. The person skilled in the art will in this case adapt the limits of the tightness and the pressure resistance of the reactor to the respective requirements based on his expertise. Preferably, in the reactor comprising the mixture of alkyl sulfide of the formula (II), optionally phase transfer catalyst, preferably the quaternary ammonium salt of the formula (IV), optionally water, and from 0.01 to 0.05 mol, preferably 0.02 to 0.04 mol, of base, preferably alkali metal hydroxide, based on 1.0 mol of alkyl sulfide of the formula (II), the pressure in the reactor at the beginning of the reaction is set to from 500 to 1500 hPa, and then alkyl halide of the formula (III) is metered in in gaseous form until the pressure in the reactor has increased by 100 to 700 hPa, preferably from 150 to 300 hPa, compared to the starting pressure. Thus, a partial pressure of alkyl halide of the formula (III)—measured in the gaseous space above the liquid reaction mixture inside the reactor—of 100 to 1500 hPa, preferably of 100 to 700 hPa, is preferably present in the reactor during the reaction. Since the alkyl halide of the formula (III) dissolves in the reaction mixture and is consumed during the reaction, the pressure in the reactor—measured in the gaseous space above the liquid reaction mixture inside the reactor—would drop without further addition of alkyl halide of the formula (III). Therefore, after the addition of base, preferably of alkali metal hydroxide, has begun, the partial pressure of alkyl halide of the formula (III) is adjusted by further metered addition of alkyl halide of the formula (III) in a range from 100 to 1500 hPa, preferably from 100 to 700 hPa. Regulating the addition of alkyl halide of the formula (III), by adjusting the elevated pressure in the reactor, ensures that sufficient alkyl halide of the formula (III) is present in the reaction system at all times during the reaction. In this preferred embodiment, measurement of the added mass of alkyl halide of the formula (III) is further used to determine when the required mass of alkyl halide of the formula (III) has been reached and the addition is then terminated.

During the preferred combined metered addition of base, preferably of alkali metal hydroxide, and alkyl halide of the formula (III), only a portion of the alkyl sulfide of the formula (II) initially charged is converted in each case to the corresponding salt of the base, preferably of the alkali metal hydroxide, and is rapidly converted to the dialkyl sulfide of the formula (I) by the alkyl halide of the formula (III) that is likewise metered in. This preferred embodiment avoids the formation of larger proportions of salt of the base, preferably of the alkali metal hydroxide, and/or of the alkyl sulfide of the formula (II) during the reaction.

Further preferred embodiments can involve the discontinuous or continuous addition of the two reactants a) base, preferably alkali metal hydroxide, and b) alkyl halide of the formula (III) to the mixture of alkyl sulfide of the formula (II) and optionally phase transfer catalyst, preferably the quaternary ammonium salt of the formula (IV), and optionally water. “Continuous addition”, according to the invention, is defined as meaning that it takes place without interruption. “Discontinuous addition”, according to the invention, means that the addition takes place with interruptions, for example in two or more discrete portions. In the case of discontinuous addition, it is possible for there to be periods of continuous addition alongside periods of discontinuous addition.

The process according to the invention is described in more detail below.

Bases that may be used are, for example, alkali metal hydroxides, such as sodium hydroxide or potassium hydroxide, alkali metal carbonates, such as sodium carbonate or potassium carbonate; alkali metal alkoxides, such as sodium alkoxides or potassium alkoxides. One or more alkali metal hydroxides, particularly preferably sodium hydroxide or potassium hydroxide, or mixtures thereof, are preferably used as base in the process according to the invention. Sodium hydroxide is especially preferably used as base. This may be, for example, sodium hydroxide in solid form, preferably as flakes or powder, or as a solution.

The amount of the base is preferably from 0.9 to 2 mol, preferably from 1.0 to 1.2 mol, based on 1 mol of alkyl sulfide of the formula (II).

The amount of the alkyl halide of the formula (III) is preferably from 0.9 to 3 mol, preferably from 1.1 to 1.5 mol, based on 1 mol of the alkyl sulfide of the formula (II).

The process according to the invention is preferably carried out without additional organic solvents, which means in the absence of organic solvents, for example ethers, for example tetrahydrofuran or 1,4-dioxane, alcohols, for example methanol or ethanol, ketones, for example acetone, esters, for example ethyl acetate, hydrocarbons, for example cyclohexane, halogenated hydrocarbons, for example dichloromethane or chlorobenzene, or nitriles, for example acetonitrile. In the process according to the invention, on the other hand, water can preferably act as solvent. Since both the reactants used, alkyl sulfide of the formula (II) and alkyl halide of the formula (III), and the product, dialkyl sulfide of the formula (I), are soluble in water only to a limited extent or not at all, the reaction takes place in two phases. The reaction mixture is therefore preferably mixed mechanically or hydraulically before, during and after addition of the alkyl halide of the formula (III) in such a way that the reaction of the reactants is at least 90 percent complete within less than 24 hours, preferably within less than 12 hours. Too little mixing would unnecessarily extend the reaction times. The amount of water added to the reaction mixture as water or solution of the base, preferably of the alkali metal hydroxide, is typically from 1 to 20 kg, preferably from 3 to 15 kg, based on 1 kg of alkyl sulfide of the formula (II).

The use of phase transfer catalysts has proven to be advantageous in the process according to the invention. Phase transfer catalysts in the context of the process according to the invention are, for example, quaternary ammonium salts such as benzyltriethylammonium bromide, benzyltrimethylammonium bromide, trihexadecylethylammonium chloride, tridodecylmethylammonium chloride, tridecylmethylammonium chloride, trioctyltrimethylammonium chloride, trioctyltrimethylammonium bromide, tetra-n-butylammonium bromide, tetraethylammonium chloride or trioctylmethylammonium bromide; and also quaternary phosphonium salts, such as hexadodecyltriethylphosphonium bromide, hexadecyltributylphosphonium chloride, or tetra-n-butylphosphonium chloride. Preference is given to using quaternary ammonium salt of the formula (IV),

R³R⁴₃N⁺Y⁻ (IV),

in which
R³ is methyl,
R⁴ is octyl and/or decyl,
X is chloride or bromide.

The amount of phase transfer catalyst, preferably quaternary ammonium salt of the formula (IV), is typically from 0.001 to 0.05 mol, preferably from 0.0015 to 0.01 mol, based on 1 mol of alkyl sulfide of the formula (II). The phase transfer catalyst, preferably quaternary ammonium salt of the formula (IV), is preferably added to the reaction mixture before base, preferably alkali metal hydroxide, and alkyl sulfide of the formula (II) are added to the reaction mixture. In addition, the phase transfer catalyst may also be prepared from simpler starting materials during or prior to the reaction.

The process according to the invention for preparing unsymmetrical dialkyl sulfides of the formula (I), comprising the reaction of an alkyl sulfide of the formula (II) with an alkyl halide of the formula (III), is preferably carried out at temperatures from 15 to 100° C., preferably from 15 to 50° C., more preferably from 25 to 40° C. If the temperature during the reaction is too low, the reaction slows down and/or the yield of dialkyl sulfide of the formula (I) decreases. If the temperature is too high, the solubility of the alkyl halide of the formula (III) in the reaction mixture decreases, which slows down the reaction and/or lowers the yield of dialkyl sulfide of the formula (I) and/or unwanted secondary components may form.

During the addition of the alkyl halide of the formula (III) to the alkyl sulfide of the formula (II), phase transfer catalyst, preferably to the quaternary ammonium salt of the formula (IV), and optionally base, preferably alkali metal hydroxide, the temperature of the reaction mixture increases due to exothermic heat development. Thus, a continuous external supply of heat to the reaction mixture can generally be dispensed with.

In one embodiment, the temperature of the reaction mixture can be maintained more quickly in the required temperature range of 15 to 100° C., preferably of 15 to 50° C., more preferably of 25 to 40° C., by means of optional external cooling. This is necessary if a faster addition of base, preferably alkali metal hydroxide, and alkyl sulfide of the formula (II) causes the temperature of the reaction mixture to rise more than desired. In an alternative embodiment, the base, preferably the alkali metal hydroxide, is added such that the required temperature range can be maintained without external cooling.

The alkyl halides of the formula (III), preferably chloromethane, bromomethane, chloroethane, bromoethane, chloropropane or bromopropane, may be added to the reaction mixture in liquid or gaseous form. The alkyl halide of the formula (III) is particularly preferably chloromethane. Chloromethane boils at −24° C. at ambient pressure and is therefore preferably introduced into the reaction mixture in gaseous form, particularly preferably relatively close to the liquid surface, for example in the range from 80 to 95% of the height of the reaction volume. If the reaction volume from the bottom of the reactor to the liquid surface, i.e. the interface between the liquid and the gaseous phase in the reactor, has a height of 1 meter, for example, the alkyl halide of the formula (III) is preferably introduced at a height of 80 to 95 cm, calculated from the lowest point of the reactor floor, i.e. 5 to 20 cm below the liquid surface. In the process according to the invention, the salt of the base with the radical X as anion is formed as by-product. Thus, if sodium hydroxide as base and an alkyl chloride as alkyl halide of the formula (III) are used in the process according to the invention, sodium chloride is formed as by-product. Due to the poor solubility, the resulting salt may precipitate directly in the vicinity of the inlet point, for example the inlet pipe, and possibly clog the opening. The person skilled in the art can determine the optimum dimensioning and positioning of the inlet point, for example the inlet pipe, by means of simple tests, depending on the shape and size of the reactor used. The only important thing here is that clogging of the inlet point, for example the inlet pipe, is prevented and the mass introduction of the alkyl halide of the formula (III) can be controlled.

The progress of the reaction can be ascertained by analyzing samples from the reaction mixture that have been worked up in the same way as the reaction mixture. The content of reactant and product can usually be determined by HPLC or gas chromatography, either as percentage by area without an external standard or as percentage by weight with an external standard.

After the reaction has ended, the product, i.e. the unsymmetrical dialkyl sulfide of the formula (I), can be separated off from the reaction mixture by terminating the hydraulic or mechanical mixing of the reaction mixture and optionally adjusting the pressure inside the reactor to ambient pressure. In this case, the reaction mixture separates into a first upper organic phase and into a second lower aqueous phase. The first upper organic phase usually comprises the unsymmetrical dialkyl sulfide of the formula (I) at a content of 95 to 99.9% by weight. After the second lower aqueous phase has been separated off, the first upper organic phase can be removed from the reactor. The unsymmetrical dialkyl sulfide of the formula (I) is obtained in yields of at least 95 percent of theory. The unsymmetrical dialkyl sulfide of the formula (I) thus obtained can usually be used without further processing as starting material for a further reaction, for example in an epoxidation reaction, such as the Corey-Chaykovsky reaction, as an intermediate of pharmaceutical active ingredients, for example fluconazole, or active ingredients in plant protection, for example cyproconazole. If the unsymmetrical dialkyl sulfide of the formula (I) has to be produced in a higher purity, the purification may be effected, for example, by distillation.

Surprisingly, a simple, cost-effective and safe process has hereby been provided with which, according to the invention, unsymmetrical dialkyl sulfides of the formula (I) are obtained in high purities and yields from an alkyl sulfide of the formula (II) and an alkyl halide of the formula (III), and which does not have the disadvantages of the prior art.

### EXAMPLES

### Example 1: Butyl methyl sulfide (inventive)

5500 g (59.76 mol) of butanethiol were initially charged in a reactor at 25° C. 7625 g of water and 60.1 g (0.13 mol) of Aliquat® 336 (mixture of N-methyl-N,N,N-trioctylammonium chloride and N-methyl-N,N,N-tridecylammonium chloride) were added thereto, whereby a biphasic mixture was generated. 250 g of aqueous sodium hydroxide solution (50% by weight, 3.14 mol) were then first added to the mixture with mixing, the temperature being kept between 25 and 40° C. 3320 g (65.76 mol) of chloromethane were then metered into the closed reactor at an initial pressure of 500 hPa below the surface of the liquid in such a way that the pressure in the reactor did not rise above 1200 hPa. Parallel to the addition of the chloromethane, 4767 g of aqueous sodium hydroxide solution (50% by weight, 59.58 mol) were metered in over the course of 3 hours, so that the temperature of the reaction mixture was in the range of 25 to 40° C. After the addition of chloromethane and sodium hydroxide solution had ended, the reaction mixture was mixed at 25 to 40° C. for a further 30 minutes. After the reaction had ended, a biphasic mixture was present in the reactor. The lower aqueous phase was first drained from the reactor and then the upper phase comprising the product was removed from the reactor. The crude product was thus obtained in an amount of 5995 g with a purity of 99% by weight (56.95 mol), which corresponds to a yield of 95% of theory.

1. A process for preparing unsymmetrical dialkyl sulfides of the formula (I),
R¹—S—R² (I),
comprising the reaction of an alkyl sulfide of the formula (II),
H—S—R² (II),
with an alkyl halide of the formula (III),
R¹—X (III),
wherein in the formulae (I), (II) and/or (III)
R¹ is a C₁—C₃-alkyl radical,
R² is a C₄—O₁₂ alkyl radical,
X is halogen,
at least in the presence of a base,
wherein a reaction mixture is formed, and the temperature of the reaction mixture during the reaction is in a range from 15 to 100° C..

2. The process as claimed in claim 1, wherein the reaction is carried out in the presence of water and a phase transfer catalyst comprising a quaternary ammonium salt of the formula (IV),
R³R⁴₃N⁺Y⁻ (IV),
in which
R³ is hydrogen or methyl,
R⁴ is a C₄—C₁₂ alkyl radical,
X is halogen.

3. The process as claimed in claim 1, wherein R² in formula (I) and formula (II) is n-butyl, n-pentyl, n-hexyl, n-heptyl, n-octyl, n-nonyl, n-decyl, n-undecyl or n-dodecyl.

4. The process as claimed in claim 1, wherein the alkyl halide of the formula (III) is used in an amount from 0.9 to 3 molar equivalents, based on the alkyl sulfide of the formula (II).

5. The process as claimed in claim 2, to wherein the phase transfer catalyst is used in an amount from 0.001 to 0.05 mol.

6. The process as claimed in claim 1, wherein the base is used in an amount from 0.9 to 2 molar equivalents, based on the alkyl sulfide of the formula (II).

7. The process as claimed in claim 1, wherein the base is sodium hydroxide or potassium hydroxide.

8. The process as claimed in claim 2, wherein to a mixture of the alkyl sulfide of the formula (II) and the phase transfer catalyst, and optionally water, is simultaneously added base, and alkyl halide of the formula (III).

9. The process as claimed in claim 2, wherein the reaction of the alkyl halide of the formula (III) and the alkyl sulfide of the formula (II) in the presence of base takes place in such a way that at the beginning of the reaction, from 0.01 to 0.05 mol of base, based on 1.0 mol of alkyl sulfide of the formula (II), is added to the mixture at least comprising alkyl sulfide of the formula (II), optionally phase transfer catalyst and optionally water, before alkyl halide of the formula (III) is added to the reaction mixture.

10. The process as claimed in claim 1, wherein the reaction takes place in a closed reactor.

11. The process as claimed in claim 10, wherein the partial pressure of alkyl halide of the formula (III) in the gaseous space above the liquid reaction mixture within the closed reactor is from 100 to 1500 hPa.

12. The process as claimed in claim 1, wherein the reaction of the alkyl halide of the formula (III) and the alkyl sulfide of the formula (II) takes place in the absence of organic solvents.
